(19) United States
(12) Patent Application Publication
(10) Pub. No.: US 2018/0356402 A1
(43) Pub. Date: Dec. 13, 2018

(54) URINE BIOMARKERS FOR DETECTING GRAFT REJECTION

(71) Applicants: The Cleveland Clinic Foundation, Cleveland, OH (US); THE UAB RESEARCH FOUNDATION, Birmingham, OH (US)

(72) Inventors: Robert L. Fairchild, Mayfield Village, OH (US); Roslyn Bernstein Mannon, Birmingham, AL (US)

(21) Appl. No.: 16/004,055

(22) Filed: Jun. 8, 2018

Related U.S. Application Data
(60) Provisional application No. 62/516,907, filed on Jun. 8, 2017.

(51) Int. Cl. G01N 33/50; G01N 33/68

(52) U.S. Cl. CPC G01N 33/5091; C12Q 1/6809; G01N 33/6893

(57) ABSTRACT

Provided herein are compositions, systems, kits, and methods for detecting rejection, or an elevated risk of rejection, of an organ or tissue graft (e.g., kidney graft) in a subject by detecting one, or a panel of, RNA markers in a urine sample from the subject.

The present application claims priority to U.S. Provisional application 62/516,907 filed Jun. 8, 2017, which is herein incorporated by reference in its entirety.

### FIELD

Provided herein are compositions, systems, kits, and methods for detecting rejection, or an elevated risk of rejection, of an organ or tissue graft (e.g., kidney graft) in a subject by detecting one, or a panel of, RNA markers in a urine sample from the subject.

### BACKGROUND

Kidney disease and failure are common medical problems associated with many illnesses. One in three Americans is considered to be at risk for kidney disease. The predictors include age above 60, diabetes, high blood pressure, and family history of kidney disease. Kidney disease can be chronic (ongoing over years with slowly declining kidney function) or acute (sharp decline in kidney function, either nearly immediate or within months). When kidney function declines to a level of reduced effectiveness, this leaves the patient with only 10-15% of kidney function remaining, giving a diagnosis of kidney failure to the patient. Common treatments for kidney failure include dialysis and kidney transplants. In 2014, about 17,000 kidney transplants occurred in the US. Of these, 11,570 kidneys came from deceased donors and 5,537 came from living donors. Typically, within the first year of a kidney transplant, there is a 7% failure rate, and within 10 years of a transplant, there is a 45% failure rate. In addition, kidney supply is not fulfilling kidney demand leading to patient death due to not receiving a transplant in time. In 2014, nearly 5,000 patients died while waiting for a transplant, on a waiting list, and more than 3,600 became too sick to receive a transplant.

Transplanted kidneys fail for various reasons, including kidney rejection. Rejection can be acute, very soon after surgery, and it occurs in about 15% of cases. There is also chronic rejection, which takes place over longer periods of time, generally taking years. Most patients who experience kidney rejection do not lose the transplanted kidney but instead go on to other treatments to manage the immune system's rejection. Accurate, early detection of the rejection can lead to better early intervention and treatment of patients. In addition, successful transplants are less expensive than dialysis, in most cases, and lead patients to live longer, healthier lives. Each year one in five transplants is performed into a patient who had previously received a transplant and is either suffering from kidney failure or kidney transplant rejection. For patients that experience transplant rejection and are unable to get another organ, they receive dialysis at a cost of $70,000 per year in the US.

Effective monitoring of kidney transplant patients for rejection is a continuing problem. As of 2016, the most effective methods of testing for transplanted kidney failure are generally considered to be through a combination of monitoring non-specific byproducts of kidney rejection, such as creatinine levels. Physicians further make a clinical assessment of a variety of patient symptoms commonly associated with kidney transplant rejection, including flu-like symptoms, chills, body aches, nausea, cough, shortness of breath, and a general sense of unease, 10 followed by or in concert with a biopsy of the kidney. Kidney biopsies are not considered a viable option for continuous, regular monitoring, and efforts to determine kidney rejection through less invasive means, including blood and urine testing, are continuously increasing. Although there have been various blood and urine tests developed during the last several years, as of March 2016, there does not seem to be a test that has been adopted for widespread clinical use. This is in part because kidney rejection is believed to be a heterologous process with many causes, depending on compatibility of the donor and recipient, and other reasons as well. As a result, some in the medical community do not believe a uniform, accurate test is possible. Monitoring of the different byproducts of kidney disease, such as serum creatinine antibodies in the blood have been attempted through blood and urine tests, but none appears to have been generally adopted as a new gold standard by the medical community. None in use so far has been established to detect kidney rejection across all types of transplant patients. A combination of creatinine monitoring and biopsy is a frequently used approach in medical environments. Acute cellular rejection is a significant type of kidney organ rejection that requires further treatment to the patient to compensate for rejection, or, at times, removal of the rejected kidney.

### SUMMARY

Provided herein are compositions, systems, kits, and methods for detecting rejection, or an elevated risk of rejection, of an organ or tissue graft (e.g., kidney graft) in a subject by detecting one, or a panel, of RNA markers in a urine sample from the subject.

In some embodiments, provided herein are methods for detecting rejection, or an elevated risk of rejection, or an organ or tissue graft in a subject comprising: detecting an upregulated level of at least one first RNA marker (e.g., 1 . . . 10 . . . 25 . . . 35) in a sample from a subject and/or a downregulated level of at least one second RNA marker (e.g., 1 . . . 10 . . . 25 . . . 35) in said sample from said subject, and thereby detecting acute rejection and/or elevated risk of rejection of said organ or tissue graft, wherein said sample comprises urine (or other sample type, such as serum or plasma) from said subject, wherein said subject has a tissue or organ graft, wherein said at least one first RNA marker that is detected as being upregulated is selected from the group consisting of: CXCL10, CXCL9, CXCL11, PRF1, CCL5, CX3CL1, IDO1, GZMA, LAG3, CD3D, CD27, KLRK1, GZMK, MMPI, CD8A, GZMB, ETS1, CXCR6, CCL2, IL2RB, CTLA4, MSR1, FYN, ITGA6, CD3E, LCK, CD2, IRF8, CD3G, CD247, GZMH, ITK, CD38, CDK1, ZAP70, LIF, CD7, CD40, CD6, IL21R, SIGIRR, CDH1, CD81, CD74, STAT1, AXL, TBX21, COG7, TCRA, C1QB, NKG7, THBS1, C1QA, EPCAM, DPP4, HLA-DPA1, TIGIT, HLA-DPB1, CXCL13, C1R, CD5, GPR171, EFEMP1, HLA-DMA, LY9, CCL8, CD96, HAVCR2, NFATC2, LAMC2, IFNG, STAT4, APOE, C4B, CTSL, SOCS1, PVR, CCRS, CD86, TNFSF12, SIGLEC1, ST6GAL1, C3, KLRC2, CTSW, CARD11, GNLY, PSMB10, ICOS, TAP1, TNFRSF11B, ITGA1, IRF4, CD4, CD84, CSF1, SH2D1A, CD8B, CD47, ABCB1, SLAMF1, HLA-DMB, IFI35, A2M, HLA-DRA, C1QBP, SPP1, VCAM1, NELL2, FLT3LG, ITGA4, TNFRSF10B, RUNX3, SERPING1, KLRC1, NLRC5, HPRT1, IKBKE, TNF, ADA PDCD1, C1S, CCR2, TAB1, IL12RB1, IL2RA, PSEN2, LRP1, NOL7, SPN, TCF7, MFGE8, MST1R, IL10, LRRN3, PSMB9, PPIA, AGK, EDC3, CD80, PSMB8, CD276, BIRC5, TNFRSF9, TP53, ZC3H14, HLA-DRB3, ICOSLG, CD200, PIN1, EGR2, ITGAE, TRAF2, ABL1, ALCAM, IRF5, SF3A3, HRAS, NOD1, PDCD1LG2, IL16, LILRB1, and IRF3, and wherein said at least one second RNA marker that is detected as being downregulated is selected from the group consisting of: ANXA1, BASP1, BCL6, BLK, CAMP, CCL17, CCL22, CCR3, CEACAM1, CEACAM6, CFD, CR2, CSF2RB, CSF3R, CXCL1, CXCL6, CXCR1, CXCR2, FUT7, IFNL1, IL18RAP, IL19, IL1A, IL1RAP, IL1RN, IL8, ITGAX, LCN2, LILRB3, LYN, MAPK3, MEFV, MYD88, OSM, PBEF1, POLR2A, PRKCD, PTGS2, S100Al2, S100A7, S100A8, SH2B2, SH2D1B, SPINKS, TFE3, TLR1, TNFRSF10C, TNFRSF11A, TNFRSF1A, TNFSF14, ADAM9, ARG2, BCL10, CASP3, CD46, CFI, DMBT1, DOCK9, DUSP4, IFI27, IKBKB, IL23A, ISG15, ITCH, LGALS3, MAPK8, MUC1, NOS2A, SERPINA3, SERPINB2, SYT17, and TREM1.

In certain embodiments, the subject, at the time the sample was obtained, is taking a first immunosuppressant. In particular embodiments, the method further comprises the step of performing at least one of the following: i) treating said subject with a second immunosuppressant that is different from said first immunosuppressant; ii) generating and/or transmitting a report that indicates said at least one first RNA maker is upregulated and/or said at least one second RNA marker is downregulated in said sample, and that said subject is in need of an immunosuppressant different from said first immunosuppressant and/or is in need of a having said tissue and/or organ graft removed (and/or in need of having said tissue or graft replaced with a different tissue or graft); iii) generating and/or transmitting a report that indicates said at least one first RNA maker is upregulated and/or said at least one second RNA marker is downregulated in said sample, and that said subject has acute rejection and/or elevated risk of rejection of said organ or tissue graft; and iv) characterizing said subject as having acute rejection and/or an elevated risk of rejection of said organ or tissue graft, based on finding that said at least one first RNA maker is upregulated and/or said at least one second RNA marker is downregulated in said sample.

In particular embodiments, the organ or tissue graft comprises a kidney or kidney tissue graft. In further embodiments, the subject at the time of collection of said sample was being treated with, and/or wherein said sample comprises, an immunosuppressant protein that comprises an extracellular domain of CTLA-4. In further embodiments, the immunosuppressant protein further comprises an Fc region. In other embodiments, the immunosuppressant protein comprises Belatacept or Abatacept. In other embodiments, the detecting at least one of said first and/or said at least one second RNA marker comprises detecting at least two (e.g., 2 . . . 5 . . . 10 . . . 15 . . . 20 . . . 25) of said first and/or second RNA markers.

In certain embodiments, the provided herein are kits, compositions, and systems comprising: a) a sample from a subject having a tissue or organ graft, wherein said sample comprises urine (or other sample type, such as serum, plasma, whole blood, etc.) from said subject; and b)reagents (e.g., sequencing reagents, nucleic acid probes, RT enzymes, polymerases, detectable labels, expression panels, NANOSTRINGS reagents, etc.) configured for detecting the level of at least one RNA marker selected from the group consisting of: CXCL10, CXCL9, CXCL11, PRF1, CCL5, CX3CL1, IDO1, GZMA, LAG3, CD3D, CD27, KLRK1, GZMK, MMP7, CD8A, GZMB, ETS1, CXCR6, CCL2, IL2RB, CTLA4, MSR1, FYN, ITGA6, CD3E, LCK, CD2, IRF8, CD3G, CD247, GZMH, ITK, CD38, CDK1, ZAP70, LIF, CD7, CD40, CD6, IL21R, SIGIRR, CDH1, CD81, CD74, STAT1, AXL, TBX21, COG7, TCRA, C1QB, NKG7, THBS1, C1QA, EPCAM, DPP4, HLA-DPA1, TIGIT, HLA-DPB1, CXCL13, C1R, CD5, GPR171, EFEMP1, HLA-DMA, LY9, CCL8, CD96, HAVCR2, NFATC2, LAMC2, IFNG, STAT4, APOE, C4B, CTSL, SOCS1, PVR, CCRS, CD86, TNFSF12, SIGLEC1, ST6GAL1, C3, KLRC2, CTSW, CARD11, GNLY, PSMB10, ICOS, TAP1, TNFRSF11B, ITGA1, IRF4, CD4, CD84, CSF1, SH2D1A, CD8B, CD47, ABCB1, SLAMF1, HLA-DMB, IFI35, A2M, HLA-DRA, C1QBP, SPP1, VCAM1, NELL2, FLT3LG, ITGA4, TNFRSF10B, RUNX3, SERPING1, KLRC1, NLRC5, HPRT1, IKBKE, TNF, ADA PDCD1, C1S, CCR2, TAB1, IL12RB1, IL2RA, PSEN2, LRP1, NOL7, SPN, TCF7, MFGE8, MST1R, IL10, LRRN3, PSMB9, PPIA, AGK, EDC3, CD80, PSMB8, CD276, BIRC5, TNFRSF9, TP53, ZC3H14, HLA-DRB3, ICOSLG, CD200, PIN1, EGR2, ITGAE, TRAF2, ABL1, ALCAM, IRF5, SF3A3, HRAS, NOD1, PDCD1LG2, IRF3, ANXA1, BASP1, BCL6, BLK, CAMP, CCL17, CCL22, CCR3, CEACAM1, CEACAM6, CFD, CR2, CSF2RB, CSF3R, CXCL1, CXCL6, CXCR1, CXCR2, FUT7, IFNL1, IL18RAP, IL19, IL1A, IL1RAP, IL1RN, IL8, ITGAX, LCN2, LILRB3, LYN, MAPK3, MEFV, MYD88, OSM, PBEF1, POLR2A, PRKCD, PTGS2, S100A12, S100A7, S100A8, SH2B2, SH2D1B, SPINKS, TFE3, TLR1, TNFRSF10C, TNFRSF11A, TNFRSF1A, TNFSF14, ADAM9, ARG2, BCL10, CASP3, CD46, CFI, DMBT1, DOCKS, DUSP4, IFI27, IKBKB, IL23A, ISG15, ITCH, LGALS3, MAPK8, MUC1, NOS2A, SERPINA3, SERPINB2, SYT17, IL16, LILRB1, and TREM1.

In some embodiments, said urine sample is from a subject having a kidney tissue or organ graft. In certain embodiments, the subject at the time of collection of said sample was being treated with, and/or wherein said sample comprises, an immunosuppressant protein that comprises an extracellular domain of CTLA-4. In further embodiments, the immunosuppressant protein further comprises an Fc region. In additional embodiments, the immunosuppressant protein comprises Belatacept or Abatacept.

In some embodiments, provided herein are methods of detecting rejection, or an elevated risk of rejection, of an organ or tissue graft in a subject comprising: detecting an upregulated level of at least one first RNA marker (e.g., 1 . . . 10 . . . 25 . . . 35) in a sample from a subject and/or a downregulated level of at least one second RNA marker (e.g., 1 . . . 10 . . . 25 . . . 35) in said sample from said subject, and thereby detecting acute rejection and/or elevated risk of rejection of said organ or tissue graft, wherein said sample comprises urine (or other sample, such as blood, plasma, whole blood, or tissue biopsy) from said subject, wherein said subject has a tissue or organ graft, wherein said at least one first RNA marker that is detected as being upregulated is selected from the group consisting of: TNFRSF11B, ETS1, CXCL10, CDK1, CXCL9, SIGIRR, CD274, CXCL11, ITGA6, and SF3A3, and wherein said at least one second RNA marker that is detected as being downregulated, are selected from the group consisting of: TNFSF4, ATG16L1, MERTK, DHX16, CXCL14, IL19, CCL17, ATF1, CFD, IFIT1, CD163, ELK1, DPP4, and LAMP2.

In particular embodiments, the subject was taking a first immunosuppressant at the time said sample was obtained. In further embodiments, the methods further comprise the step of performing at least one of the following: i) treating said subject with a second immunosuppressant that is different from said first immunosuppressant; ii) generating and/or transmitting a report that indicates said at least one first RNA maker is upregulated and/or said at least one second RNA marker is downregulated in said sample, and/or that said subject is in need of an immunosuppressant different from said first immunosuppressant, and/or is in need of a having said tissue and/or organ graft removed or replaced; iii) generating and/or transmitting a report that indicates said at least one first RNA maker is upregulated and/or said at least one second RNA marker is downregulated in said sample, and/or that said subject has acute rejection and/or elevated risk of rejection of said organ or tissue graft; and iv) characterizing said subject as having acute rejection and/or an elevated risk of rejection of said organ or tissue graft, based on finding that said at least one first RNA maker is upregulated and/or said at least one second RNA marker is downregulated in said sample.

In certain embodiments, said organ or tissue graft comprises a kidney or kidney tissue graft. In further embodiments, the subject has been treated with an anti-CD52 monoclonal antibody or antigen binding fragment thereof. In additional embodiments, the anti-CD52 monoclonal antibody comprises Alemtuzumab/CAMPATH. In some embodiments, the subject at the time of collection of said sample was being treated with, and/or wherein said sample comprises, an immunosuppressant comprising tacrolimus (aka FK506). In some embodiments, the at least one of said first and/or second RNA marker comprises detecting at least two (e.g., 2 . . . 5 . . . 10 . . . 15 . . . 25 . . . 35) of said first and/or second RNA markers. In other embodiments, the detecting at least one of said first and/or second RNA marker comprises detecting at least four of said first and/or second RNA markers.

In some embodiments, provided herein are compositions, kits, or systems comprising: a) a sample from a subject having a tissue or organ graft, wherein said sample comprises urine from said subject; and b) reagent configured for detecting the level of at least one RNA marker selected from the group consisting of: CCL17, CD163, CD274, CDK1, CFD, CXCL10, CXCL11, CXCL14, CXCL9, DHX16, DPP4, ELK1, ETS1, IFIT1, IL19, ITGA6, LAMP2, MERTK, SF3A3, SIGIRR, TNFRSF11B, and TNFSF4.

In certain embodiments, the urine sample is from a subject having a kidney tissue or organ graft. In further embodiments, the subject has been treated with an anti-CD52 monoclonal antibody or antigen binding fragment thereof for immunosuppression of said tissue or graft. In other embodiments, the anti-CD52 monoclonal antibody comprises Alemtuzumab/CAMPATH. In other embodiments, the subject at the time of collection of said sample was being treated with, and/or wherein said sample comprises, an immunosuppressant comprising tacrolimus (aka FK506).

In some embodiments, provided herein are methods of detecting rejection or BK virus infection (e.g., distinguishing between acute rejection and BK virus infection), or an elevated risk of rejection, of an organ or tissue graft in a subject comprising: detecting an upregulated level of at least one first RNA marker in a sample from a subject and/or a downregulated level of at least one second RNA marker in said sample from said subject, and thereby detecting acute rejection and/or elevated risk of rejection of said organ or tissue graft, wherein said sample comprises urine from said subject, wherein said subject has a tissue or organ graft, wherein said at least one first RNA marker that is detected as being upregulated and is selected from the group consisting of: SIGLEC1, CD163, PSEN1, DPP4, CEACAM8, PYCARD, MTMR14, ICOSLG, NOL7, C2, HLA.DRB3, CD68, ST6GAL1, SLAMF1, MERTK, PRAME, ELK1, STAT4, CD99, CD86, LAMP2, ITGB1, IL21R, IL6ST, TNF, GPATCH3, GPI, ATF1, PIN1, and YTHDF2, and wherein said at least one second RNA marker that is detected as being downregulated, is selected from the group consisting of: HMGB1, MAPK9, SPA17, TNFRSF11B, GTF3C1, BST2, PRPF38A, MAF, BATF, EGFR, CD274, CCL17, SERPINA3, GBP1, and PBEF1.

In certain embodiments, the BK virus infection is detected (and distinguished from acute rejection). In other embodiments, acute rejection is detected (e.g., and distinguished from BK virus infection). In some embodiments, the organ or tissue graft comprises a kidney or kidney tissue graft. In additional embodiments, the subject has been treated with an anti-CD52 monoclonal antibody or antigen binding fragment thereof. In some embodiments, the anti-CD52 monoclonal antibody comprises Alemtuzumab/CAMPATH. In certain embodiments, said subject at the time of collection of said sample was being treated with, and/or wherein said sample comprises, an immunosuppressant comprising tacrolimus (aka FK506).

In some embodiments, provided herein are kits, compositions, and systems comprising: a) a sample from a subject having a tissue or organ graft, wherein said sample comprises urine from said subject; and b) reagent configured for detecting the level of at least one RNA marker selected from the group consisting of: ATF1, BATF, BST2, C2, CCL17, CD163, CD274, CD68, CD86, CD99, CEACAM8, DPP4, EGFR, ELK1, GBP1, GPATCH3, GPI, GTF3C1, HLA.DRB3, HMGB1, ICOSLG, IL21R, IL6ST, ITGB1, LAMP2, MAF, MAPK9, MERTK, MTMR14, NOL7, PBEF1, PIN1, PRAME, PRPF38A, PSEN1, PYCARD, SERPINA3, SIGLEC1, SLAMF1, SPA17, ST6GAL1, STAT4, TNF, TNFRSF11B, and YTHDF2.

In certain embodiments, the urine sample is from a subject having a kidney tissue or organ graft. In further embodiments, the subject has been treated with an anti-CD52 monoclonal antibody or antigen binding fragment thereof. In additional embodiments, the anti-CD52 monoclonal antibody comprises Alemtuzumab/CAMPATH. In some embodiments, the subject at the time of collection of said sample was being treated with, and/or wherein said sample comprises, an immunosuppressant comprising tacrolimus (aka FK506).

In some embodiments, provided herein are methods of detecting rejection, or an elevated risk of rejection, or an organ or tissue graft in a subject comprising: detecting an upregulated level of at least one first RNA marker (e.g., mRNA sequence or portion thereof) in a sample from a subject and/or a downregulated level of at least one second RNA marker (e.g., mRNA sequence or portion thereof) in the sample from the subject, and thereby detecting acute rejection and/or elevated risk of rejection of the organ or tissue graft, wherein the sample comprises urine from the subject, wherein the subject has a tissue or organ graft, wherein the first RNA marker (e.g., mRNA sequence or portion thereof) that is detected as being upregulated is selected from the group consisting of: CXCL10, CXCL9, CXCL11, PRF1, CCL5, CX3CL1, IDO1, GZMA, LAG3, CD3D, CD27, KLRK1, GZMK, MMP7, CD8A, GZMB, ETS1, CXCR6, CCL2, IL2RB, CTLA4, MSR1, FYN, ITGA6, CD3E, LCK, CD2, IRF8, CD3G, CD247, GZMH, ITK, CD38, CDK1, ZAP70, LIF, CD7, CD40, CD6, IL21R, SIGIRR, CDH1, CD81, CD74, STAT1, AXL, TBX21, COG7, TCRA, C1QB, NKG7, THBS1, C1QA, EPCAM, DPP4, HLA-DPA 1, TIGIT, HLA-DPB1, CXCL13, C1R, CD5, GPR171, EFEMP1, HLA-DMA, LY9, CCL8, CD96, HAVCR2, NFATC2, LAMC2, IFNG, STAT4, APOE, C4B, CTSL, SOCS1, PVR, CCRS, CD86, TNFSF12, SIGLEC1, ST6GAL1, C3, KLRC2, CTSW, CARD11, GNLY, PSMB10, ICOS, TAP1, TNFRSF11B, ITGA1, IRF4, CD4, CD84, CSF1, SH2D1A, CD8B, CD47, ABCB1, SLAMF1, HLA-DMB, IFI35, A2M, HLA-DRA, C1QBP, SPP1, VCAM1, NELL2, FLT3LG, ITGA4, TNFRSF10B, RUNX3, SERPING1, KLRC1, NLRC5, HPRT1, IKBKE, TNF, ADA, PDCD1, C 1 S, CCR2, TAB1, IL12RB1, IL2RA, PSEN2, LRP1, NOL7, SPN, TCF7, MFGE8, MST1R, IL10, LRRN3, PSMB9, PPIA, AGK, EDC3, CD80, PSMB8, CD276, BIRC5, TNFRSF9, TP53, ZC3H14, HLA-DRB3, ICOSLG, CD200, PIN1, EGR2, ITGAE, TRAF2, ABL1, ALCAM, IRF5, SF3A3, HRAS, NOD1, PDCD1LG2, and IRF3; and wherein the second RNA marker (e.g., mRNA sequence or portion thereof) that is detected as being downregulated is selected from the group consisting of: ANXA1, BASP1, BCL6, BLK, CAMP, CCL17, CCL22, CCR3, CEACAM1, CEACAM6, CFD, CR2, CSF2RB, CSF3R, CXCL1, CXCL6, CXCR1, CXCR2, FUT7, IFNL1, IL18RAP, IL19, IL1A, IL1RAP, IL1RN, IL8, ITGAX, LCN2, LILRB3, LYN, MAPK3, MEFV, MYD88, OSM, PBEF1, POLR2A, PRKCD, PTGS2, S100A12, S100A7, S100A8, SH2B2, SH2D1B, SPINKS, TFE3, TLR1, TNFRSF10C, TNFRSF11A, TNFRSF1A, TNFSF14, and TREM1.

In particular embodiments, provided herein are methods of detecting rejection, or an elevated risk of rejection, or an organ or tissue graft in a subject comprising: detecting an upregulated level of at least one RNA marker in a sample from a subject, and thereby detecting acute rejection and/or elevated risk of rejection of the organ or tissue graft, wherein the sample comprises urine from the subject, wherein the subject has a tissue or organ graft, wherein the RNA marker that is detected as being upregulated is selected from the group consisting of: CXCL9, CXCL10, CXCL11, LAG3, CD38, CD3D, IDO1, CCL5, PRF1, KLRK1, TCRA, CTLA4, CD8A, STAT4, and CD27.

In certain embodiments, provided herein are methods of detecting rejection, or an elevated risk of rejection, or an organ or tissue graft in a subject comprising: detecting an upregulated level of at least one RNA marker in a sample from a subject, and thereby detecting acute rejection and/or elevated risk of rejection of the organ or tissue graft, wherein the sample comprises urine from the subject, wherein the subject has a tissue or organ graft, wherein the RNA marker that is detected as being upregulated is selected from the group consisting of: C1QA, C1QB, C1R, CD3E, CTSL, CX3CL1, GSMA, HAVCR2, IF135, MSR1, SIGLEC1, and ENG.

In some embodiments, the organ or tissue graft comprises a kidney or kidney tissue graft or a liver or liver tissue. In particular embodiments, the detecting at least one of the first and/or second RNA marker comprises detecting at least two, three, four, five, six, seven, . . . twenty . . . thirty . . . forty . . . or seventy-five of the first and/or second RNA markers. In certain embodiments, a Nanostrings assay is employed to detect the first and/or second RNA markers.

In particular embodiments, provided herein are kits, systems, and/or compositions comprising: a) a sample from a subject having a tissue or organ graft, wherein the sample comprises urine from the subject; and b) reagent configured for detecting the level of at least one RNA marker (e.g., mRNA sequence or portion thereof) selected from the group consisting of: CXCL10, CXCL9, CXCL11, PRF1, CCL5, CX3CL1, IDO1, GZMA, LAG3, CD3D, CD27, KLRK1, GZMK, MMP7, CD8A, GZMB, ETS1, CXCR6, CCL2, IL2RB, CTLA4, MSR1, FYN, ITGA6, CD3E, LCK, CD2, IRF8, CD3G, CD247, GZMH, ITK, CD38, CDK1, ZAP70, LIF, CD7, CD40, CD6, IL21R, SIGIRR, CDH1, CD81, CD74, STAT1, AXL, TBX21, COG7, TCRA, C1QB, NKG7, THBS1, C1QA, EPCAM, DPP4, HLA-DPA1, TIGIT, HLA-DPB1, CXCL13, C1R, CD5, GPR171, EFEMP1, HLA-DMA, LY9, CCL8, CD96, HAVCR2, NFATC2, LAMC2, IFNG, STAT4, APOE, C4B, CTSL, SOCS1, PVR, CCRS, CD86, TNFSF12, SIGLEC1, ST6GAL1, C3, KLRC2, CTSW, CARD11, GNLY, PSMB10, ICOS, TAP1, TNFRSF11B, ITGA1, IRF4, CD4, CD84, CSF1, SH2D1A, CD8B, CD47, ABCB1, SLAMF1, HLA-DMB, IFI35, A2M, HLA-DRA, C1QBP, SPP1, VCAM1, NELL2, FLT3LG, ITGA4, TNFRSF10B, RUNX3, SERPING1, KLRC1, NLRC5, HPRT1, IKBKE, TNF, ADA, PDCD1, C1S, CCR2, TAB1, IL12RB1, IL2RA, PSEN2, LRP1, NOL7, SPN, TCF7, MFGE8, MST1R, IL10, LRRN3, PSMB9, PPIA, AGK, EDC3, CD80, PSMB8, CD276, BIRC5, TNFRSF9, TP53, ZC3H14, HLA-DRB3, ICOSLG, CD200, PIN1, EGR2, ITGAE, TRAF2, ABL1, ALCAM, IRF5, SF3A3, HRAS, NOD1, PDCD1LG2, IRF3, ANXA1, BASP1, BCL6, BLK, CAMP, CCL17, CCL22, CCR3, CEACAM1, CEACAM6, CFD, CR2, CSF2RB, CSF3R, CXCL1, CXCL6, CXCR1, CXCR2, FUT7, IFNL1, IL18RAP, IL19, IL1A, IL1RAP, IL1RN, IL8, ITGAX, LCN2, LILRB3, LYN, MAPK3, MEFV, MYD88, OSM, PBEF1, POLR2A, PRKCD, PTGS2, S100Al2, S100A7, S100A8, SH2B2, SH2D1B, SPINKS, TFE3, TLR1, TNFRSF10C, TNFRSF11A, TNFRSF1A, TNFSF14, and TREM1.

In certain embodiments, provided herein are kits, systems, and/or compositions comprising: a) a sample from a subject having a tissue or organ graft, wherein the sample comprises urine from the subject; and b) reagent configured for detecting the level of at least one RNA marker selected from the group consisting of: C1QA, C1QB, C1R, CD3E, CTSL, CX3CL1, GSMA, HAVCR2, IF135, MSR1, SIGLEC1, and ENG.

In particular embodiments, provided herein are kits, systems, and/or compositions comprising: a) a sample from a subject having a tissue or organ graft, wherein the sample comprises urine from the subject; and b) reagent configured for detecting the level of at least one RNA marker selected from the group consisting of: CXCL9, CXCL10, CXCL11, LAG3, CD38, CD3D, IDO1, CCL5, PRF1, KLRK1, TCRA, CTLA4, CD8A, STAT4, and CD27. In certain embodiments, the urine sample is from a subject having a kidney tissue or organ graft.

### DESCRIPTION OF THE FIGURES

FIG. 1 shows an RNA expression profile of an RNA sample.

FIG. 2 shows an RNA expression profile of an RNA sample.

FIG. 3 shows genes upregulated at the time of acute rejection.

FIG. 4 shows an RNA expression profile of an RNA sample.

FIG. 5 shows an RNA expression profile of an RNA sample.

FIG. 6 shows an RNA expression profile of an RNA sample.

FIG. 7 shows an RNA expression profile of an RNA sample.

FIG. 8 shows an RNA expression profile of an RNA sample.

FIG. 9 shows an RNA expression profile of an RNA sample.

FIG. 10 shows an RNA expression profile of an RNA sample.

FIG. 11 shows an RNA expression profile of an RNA sample.

FIG. 12 shows an RNA expression profile of an RNA sample.

FIG. 13 shows rejection status—differential expression in ARV vs baseline of control.

FIG. 14 shows an RNA expression profile of an RNA sample.

FIG. 15 shows an RNA expression profile of an RNA sample.

FIG. 16 shows an RNA expression profile of an RNA sample.

FIG. 17 shows an RNA expression profile of an RNA sample.

### DETAILED DESCRIPTION

Provided herein are compositions, systems, kits, and methods for detecting rejection, or an elevated risk of rejection, of an organ or tissue graft (e.g., kidney graft) in a subject by detecting one, or a panel of, RNA markers in a urine sample from the subject.

In certain embodiments, the nanostring detection platform (NanoString Technologies) is employed to detect mRNA markers in a sample. The nanostring platform technology uses molecular “barcodes” and single molecule imaging to detect and count hundreds of unique transcripts in a single reaction. The resulting tests are able to identify genetic sequences at much smaller quantities and without the need for extensive amplification of any genetic or proteomic material, which saves time and money. This process has been demonstrated to yield highly accurate results.

### EXAMPLE 1

In work conducted during development of embodiments of the present disclosure, a number of different trials were conducted (e.g., CTOT-10, -15 and -16 trials). Results are presented below and in the figures.

In one trial, peripheral blood and urine RNA samples collected from 7 patients enrolled in a clinical trial who had ongoing kidney graft rejection as well as 7 transplant patients without rejection, using a 796 gene code set and the NanoString platform. We observed no distinctive signatures in the peripheral blood RNA from patients with rejection vs. the controls, but the urine RNA from the rejecting patients had 18 genes that were expressed at 5-fold or higher levels when compared to the controls. The study was expanded to further to include 11 samples in each cohort and observed a distinctive pattern (shown below) in the samples from the rejecting kidney transplant patients when compared to controls. It should be noted that the single outlier in the control group was flagged by the NanoString instrument as being of suboptimal quality and that the two outliers in the rejection samples were taken within the first month after the transplant. The results from the study confirm our initial studies that NanoString analysis of urine sediment RNA can be used to distinguish patients experiencing rejection from those that are not.

In certain embodiments, the detection methods here are used for all kidney transplant patients after the 1st month post-transplant. In some embodiments, while current diagnosis is confirmed by kidney graft biopsy, this technology could guide nephrologists in determining whether a biopsy is warranted or not.

### Background

A major goal of the Clinical Trials in Organ Transplantation (CTOT) studies is the identification of biomarkers that predict or diagnose acute graft rejection in transplant patients. Potential gene markers of graft injury have been identified in graft biopsy tissue by means of microarrays and in urine sediment RNA by quantitative PCR.

### Methods

Using Nanostring technology, we compared gene expression changes during acute rejection in tissue and urine sediment RNA from kidney transplant patients on a single platform. Study subjects were enrolled in the CTOT-10, -15 and -16 trials and were treated with either belatacept-based regimens or with conventional immunosuppression.

### Results

We measured expression of 795 immune function genes in urine sediment RNA collected from 12 subjects undergoing acute rejection and 10 control subjects with stable graft function and observed a set of 27 genes upregulated by five-fold or greater at the time of clinically diagnosed rejection. We then assessed the same 795 genes in graft tissue RNA from a second set of subjects that included 12 with acute rejection and 3 controls with no evidence of rejection. In graft tissue RNA, 159 genes were upregulated by five-fold or greater at the time of rejection. Of the genes upregulated 5-fold or more at the time of rejection, 15 were common to both graft tissue and urine sediment RNA, 144 were unique to graft tissue and 12 were unique to urine. The transcripts overexpressed in rejecting renal allograft tissue were predominantly chemokines and their receptors, immune signaling molecules, and T cell and NK cell-associated markers. A subset of these genes was also upregulated in the urine of transplant recipients undergoing acute rejection. Genes upregulated in the urine sediment but not the tissue of recipients undergoing rejection included complement components and adhesion molecules.

### Exemplary Conclusions

Our observation of common genes in the RNA of graft tissue and urine sediment supports the utility of urine as an ideal non-invasive sample site for interrogating graft status.

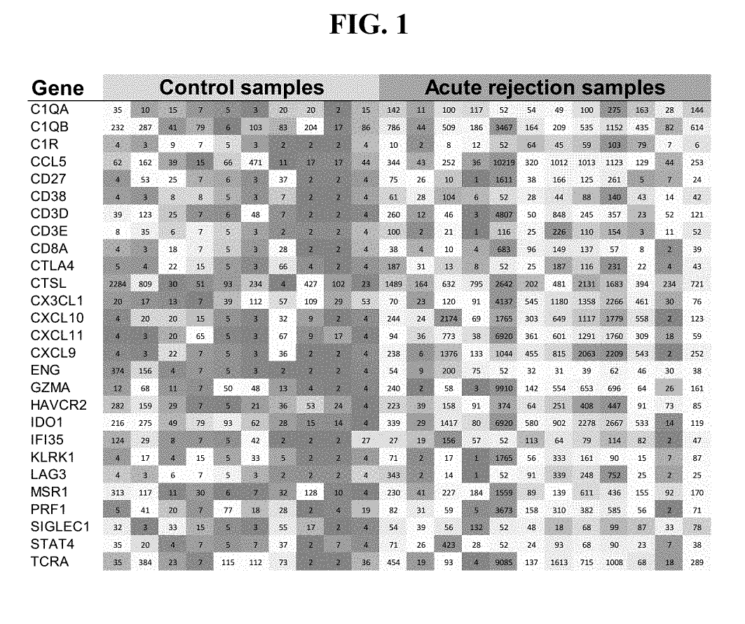

FIG. 1 shows Genes upregulated in urine sediment RNA at the time of acute rejection 27 genes were expressed at least 5-fold higher in the urine of subjects undergoing acute rejection (n=12) than in urine from control subjects with stable graft function and no evidence of rejection (n=10). The most highly upregulated genes were CXCL9 (53-fold), CXCL10 (34-fold), and CXCL11 (26-fold over controls). The remaining genes were upregulated between 5 and 10-fold over controls.

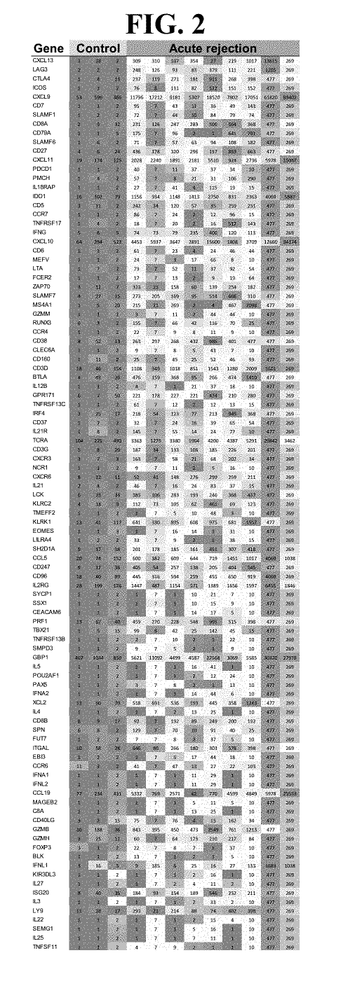

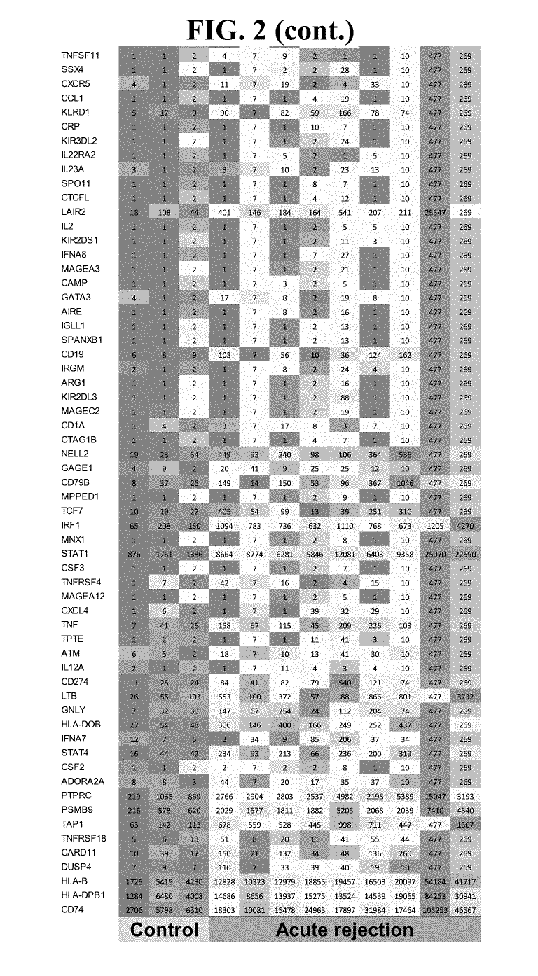

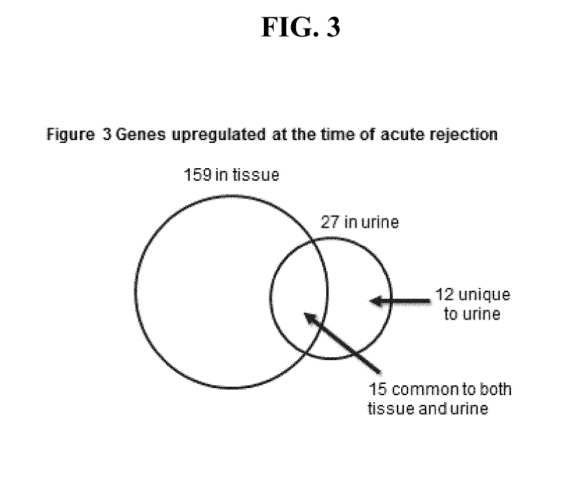

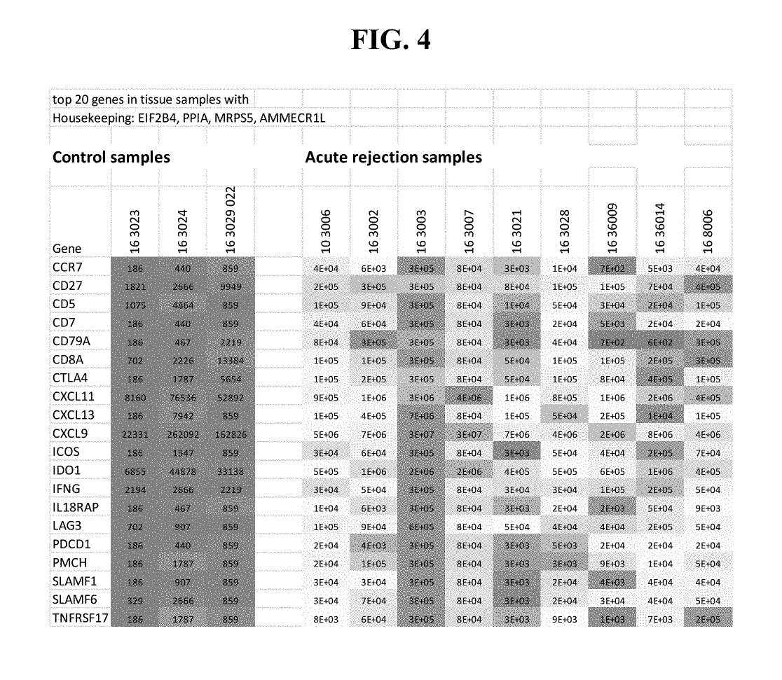

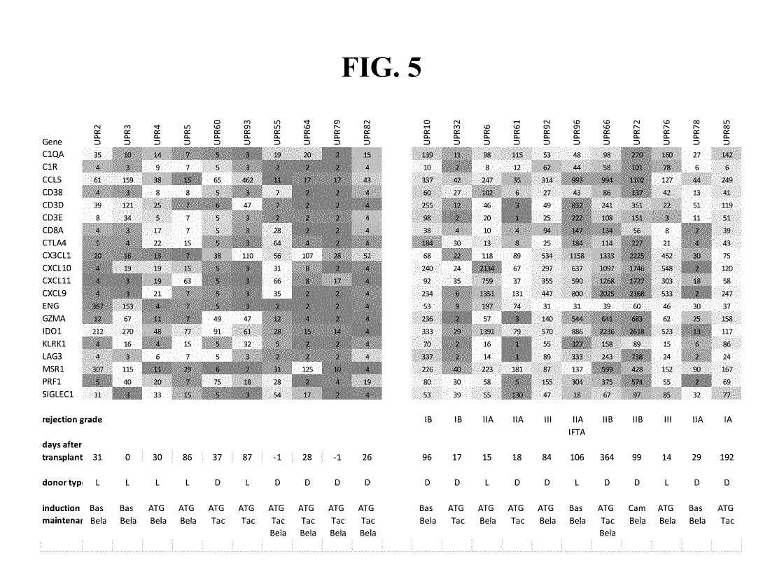

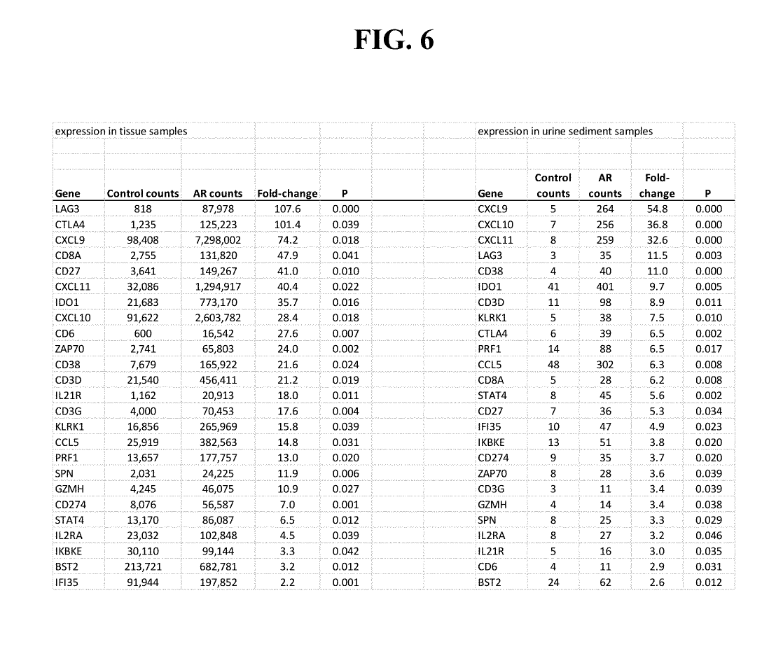

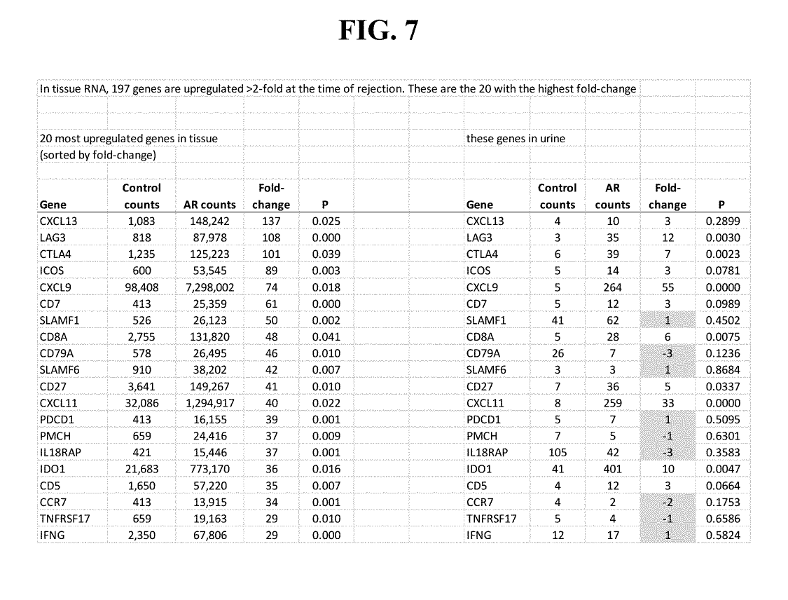

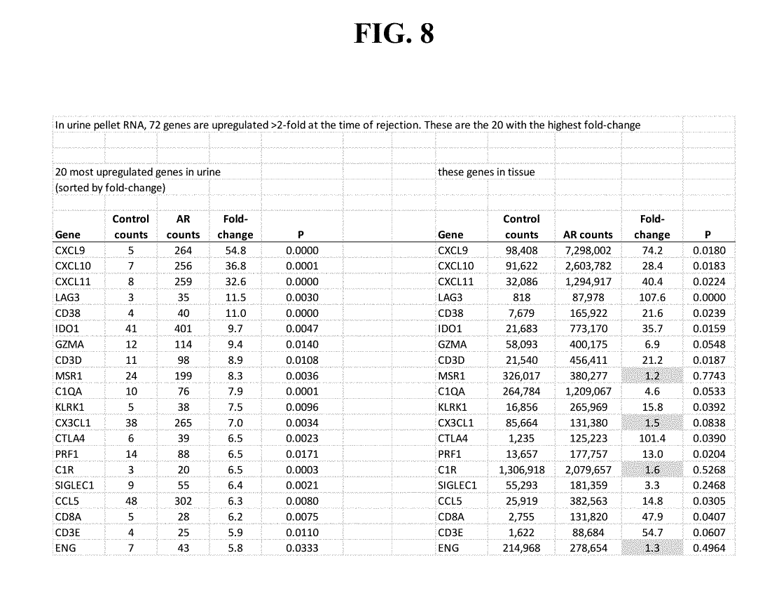

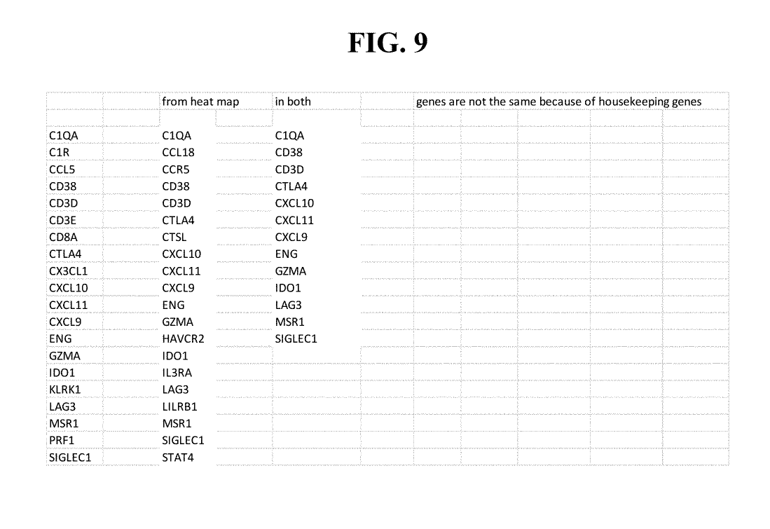

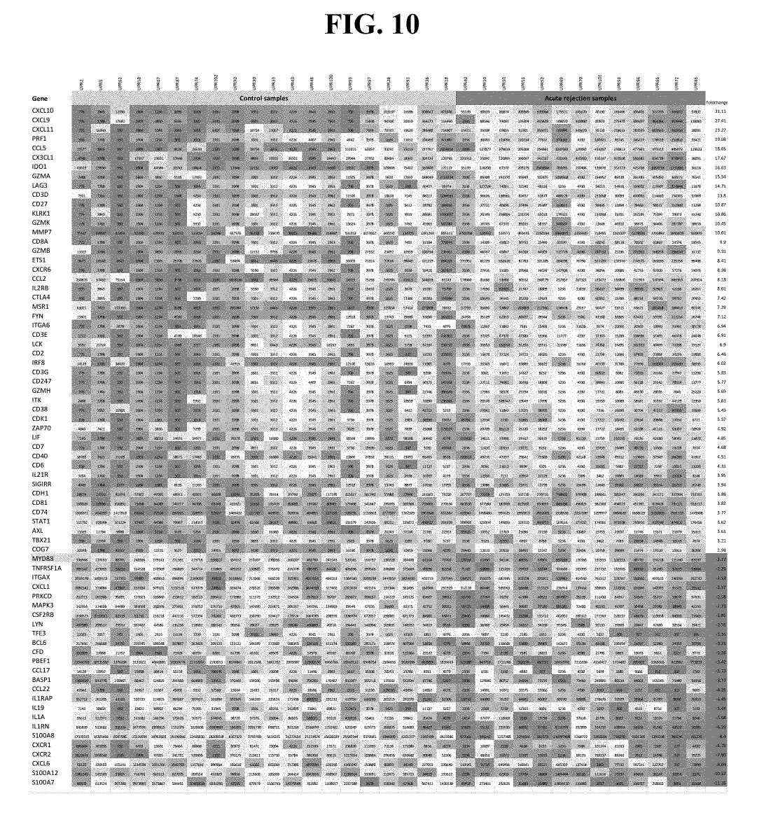

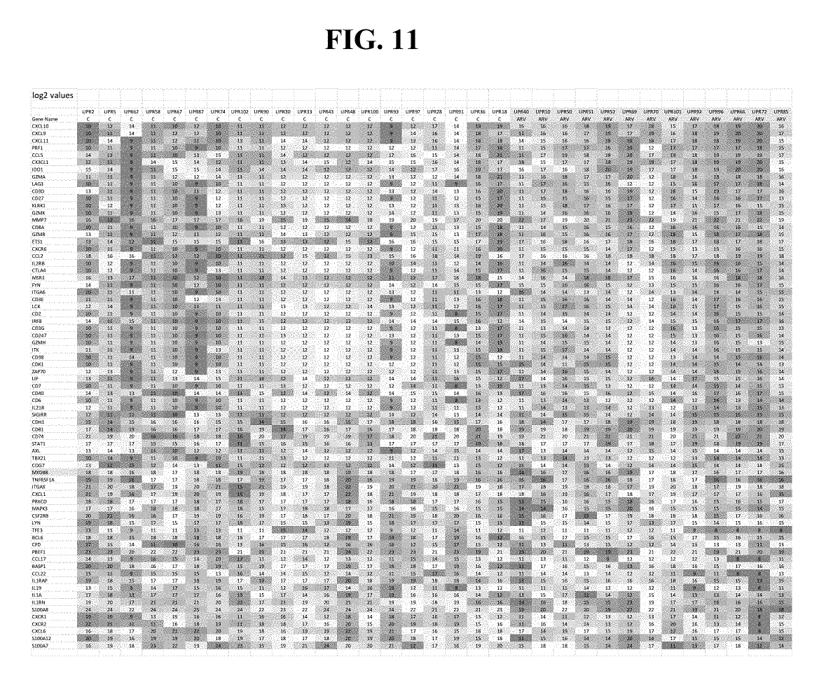

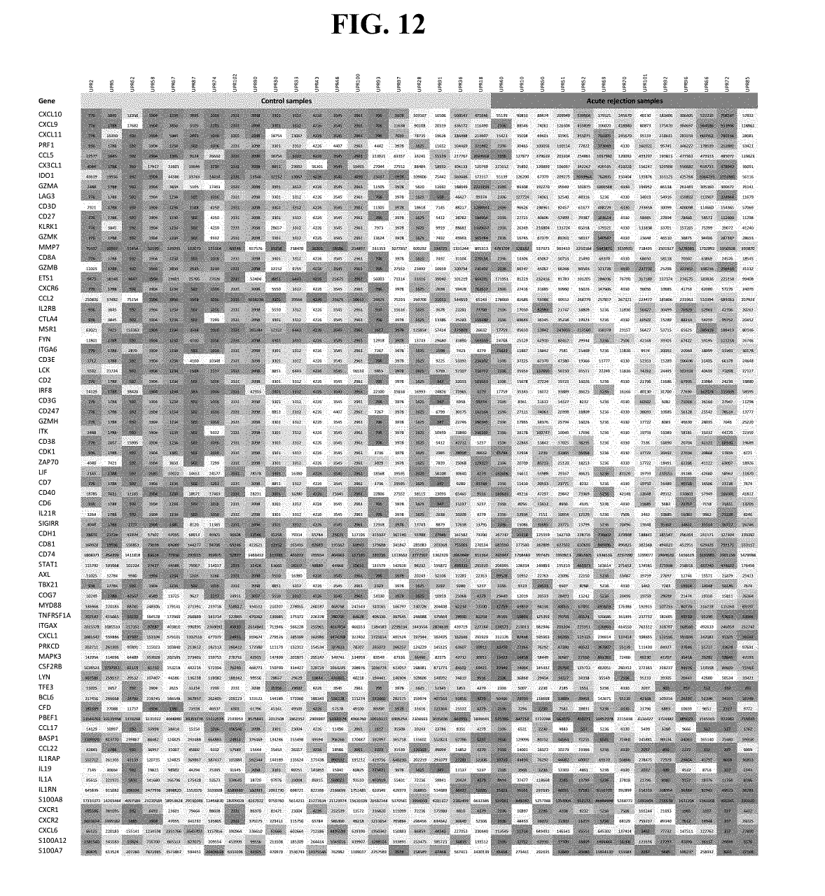

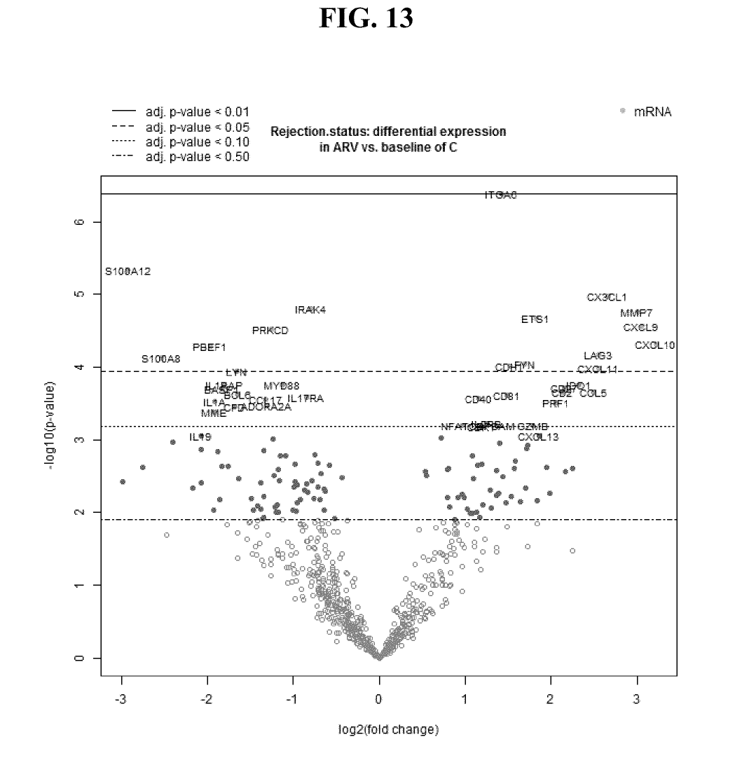

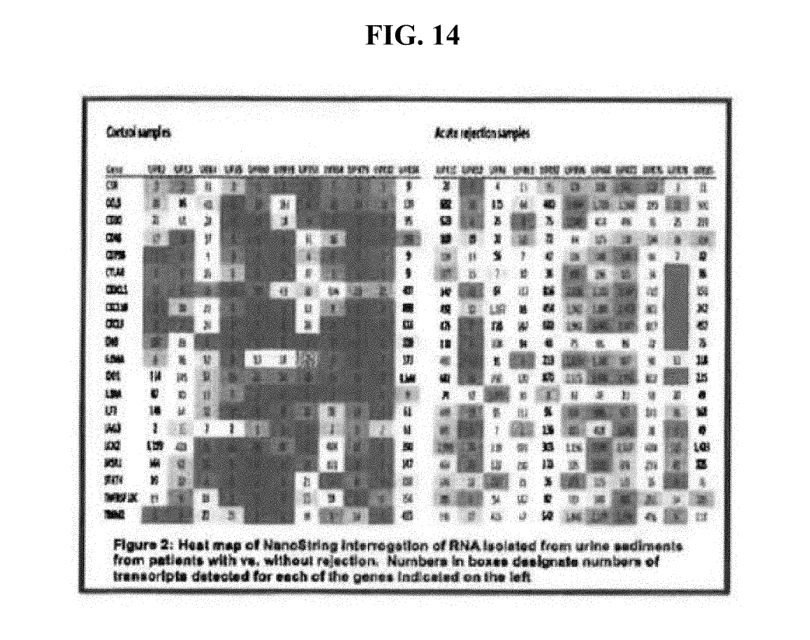

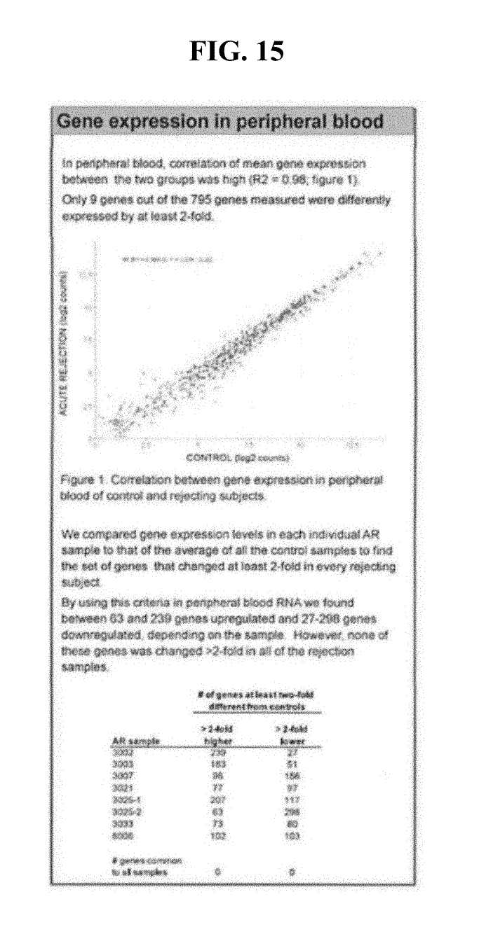

FIG. 2 shows Genes upregulated in renal biopsy tissue at the time of acute rejection 159 genes were expressed at least 5-fold higher in tissue of subjects undergoing acute rejection (n=9) when compared with tissue from control subjects (n=3). The most highly upregulated genes in tissue were CXCL13, LAG3 and CTLA4 (>100-fold higher than controls), and ICOS, CXCL9 and CD7 (>50-fold higher than controls).

Table 1 (below) shows genes upregulated in both tissue and urine at the time of acute rejection. 15 genes were upregulated by at least 5-fold in both urine and tissue samples. These common transcripts largely suggest the presence and activity of graft-infiltrating cytotoxic T cells and include chemoattractants (CXCL9, CXCL10, CXCL11, CCL5), T cell surface markers (CD3D, TCRA, CD8A, KLRK1, LAG3), co-stimulatory and activating molecules (CTLA4, CD27, CD38, STAT4), and mediators of cytotoxicity (PRF).

TABLE 1

| | | | | |
|---|---|---|---|---|
| | Control | Rejection | Fold- | |
| | samples | samples | change | |
| Gene | (C) | (AR) | AR/C | P value |

expression in urine

| | | | | |
|---|---|---|---|---|
| CXCL9 | 5 | 269 | 52.8 | 4E−05 |
| CXCL10 | 8 | 261 | 34.1 | 6E−05 |
| CXCL11 | 10 | 264 | 26.4 | 7E−05 |
| LAG3 | 3 | 35 | 10.6 | 0.004 |
| CD38 | 4 | 41 | 10.2 | 5E−07 |
| CD3D | 11 | 100 | 9.3 | 0.007 |
| IDO1 | 45 | 408 | 9.1 | 0.004 |
| CCL5 | 44 | 308 | 7.0 | 0.004 |
| PRF1 | 13 | 90 | 6.9 | 0.012 |
| KLRK1 | 6 | 39 | 6.8 | 0.01 |
| TCRA | 27 | 185 | 6.8 | 0.04 |
| CTLA4 | 7 | 40 | 5.9 | 0.002 |
| CD8A | 5 | 29 | 5.9 | 0.008 |
| STAT4 | 8 | 46 | 5.7 | 0.001 |
| CD27 | 7 | 37 | 5.3 | 0.03 |

expression in tissue

| | | | | |
|---|---|---|---|---|
| CXCL9 | 230 | 17,242 | 75.1 | 0.02 |
| CXCL10 | 214 | 6,152 | 28.8 | 0.02 |
| CXCL11 | 75 | 3,059 | 40.9 | 0.02 |
| LAG3 | 2 | 208 | 109.0 | 6E−08 |
| CD38 | 18 | 392 | 21.9 | 0.02 |
| CD3D | 50 | 1,078 | 21.5 | 0.02 |
| IDO1 | 51 | 1,827 | 36.1 | 0.01 |
| CCL5 | 60 | 904 | 15.0 | 0.03 |
| PRF1 | 32 | 420 | 13.2 | 0.02 |
| KLRK1 | 39 | 628 | 16.0 | 0.04 |
| TCRA | 225 | 4,026 | 17.9 | 0.006 |
| CTLA4 | 4 | 296 | 78.1 | 0.02 |
| CD8A | 6 | 311 | 48.5 | 0.04 |
| STAT4 | 31 | 203 | 6.6 | 0.01 |
| CD27 | 8 | 353 | 41.5 | 0.01 |

Table 2 shows genes upregulated only in urine at the time of acute rejection 12 genes were upregulated by at least 5-fold in urine sediment but NOT in biopsy tissue at the time of acute rejection. While this set contains T cell-associated transcripts (CD3E, GranzymeA), the upregulated genes unique to the urine also include complement components (C1Qa, C1Qb, C1R), adhesion molecules (SIGLEC1, ENG, CX3CL1), and genes involved in extracellular matrix reorganization (CTSL), inflammatory signaling (IFI35), and macrophage functions (MSR1, HAVCR2).

TABLE 2

| | Control | Rejection | Fold- | |
|---|---|---|---|---|
| | samples | samples | change | |
| Gene | (C) | (AR) | AR/C | P value |
| C1QA | 9 | 77 | 8.1 | 0.00004 |
| C1QB | 71 | 363 | 5.1 | 0.005 |
| C1R | 3 | 21 | 6.0 | 0.001 |
| CD3E | 5 | 26 | 5.6 | 0.01 |
| CTSL | 109 | 665 | 6.1 | 0.02 |
| CX3CL1 | 32 | 270 | 8.3 | 0.002 |
| GZMA | 12 | 116 | 9.9 | 0.01 |
| HAVCR2 | 27 | 144 | 5.3 | 0.004 |
| IFI35 | 9 | 48 | 5.1 | 0.01 |
| MSR1 | 25 | 203 | 8.2 | 0.002 |
| SIGLEC1 | 9 | 56 | 6.1 | 0.001 |
| ENG | 8 | 44 | 5.8 | 0.02 |

Gene expression changes in the urine can distinguish stable and rejecting kidney allografts. Observation of the same upregulated genes in rejecting urine and biopsies from different patients supports the use of urine as a non-invasive surrogate for biopsy in evaluating graft status

In certain embodiments, urine obtained from transplant recipients is centrifuged and the remaining urine pellet material is prepared for RNA isolation. RNA analysis may be performed an RNA assay such as the NanoString technologies nCounter analysis system. The multigene platform (codeset) included those encoding many chemokines, cytokines, interleukins and immune-cell specific markers, as well as inflammation, antigen processing and presentation, and T and B cell activation and differentiation pathways. The data as shown in FIG. 1 indicate a strong demarcation in recipients with immune activation of cellular rejection. The presently disclosed methods provide, for example, a noninvasive approach using patient biologic material with rapid turnaround. The use of this platform would provide rapid diagnosis of acute cellular rejection in a kidney transplant. Further application could be used for other solid organ transplant recipients. Additionally, this methodology could provide a non-invasive monitoring tool for patients, coupled with their clinical lab values, to assess for occult allograft injury prior to clinical injury. This includes monitoring patients during both intentional and inadvertent reductions of immunosuppression during periods of opportunistic infection or patient non adherence.

In one trial, a gene panel was employed. NanoString was employed in the CTOT consortium, where we simultaneously analyzed the express on of 795 genes in peripheral blood and urine sediment RNA prepared from 17 renal transplant subjects enrolled in the CTOT16 study. The multigene platform included those encoding many chemokines, cytokines, interleukins and immune-cell specific markers, as well as inflammation, antigen processing and presentation, and T and B cell activation and differentiation pathways. Seven samples were obtained from 7 subjects at the time of biopsy-proven acute rejection of Banff grade Ior II and from twenty-three samples from 10 subjects with stable graft function and no evidence of rejection for analysis. All of the peripheral blood samples were of high quality (RIN>7) whereas several urine RNA samples were degraded to varying degrees and only 6 of the 7 samples were deemed of sufficient quality for analysis.

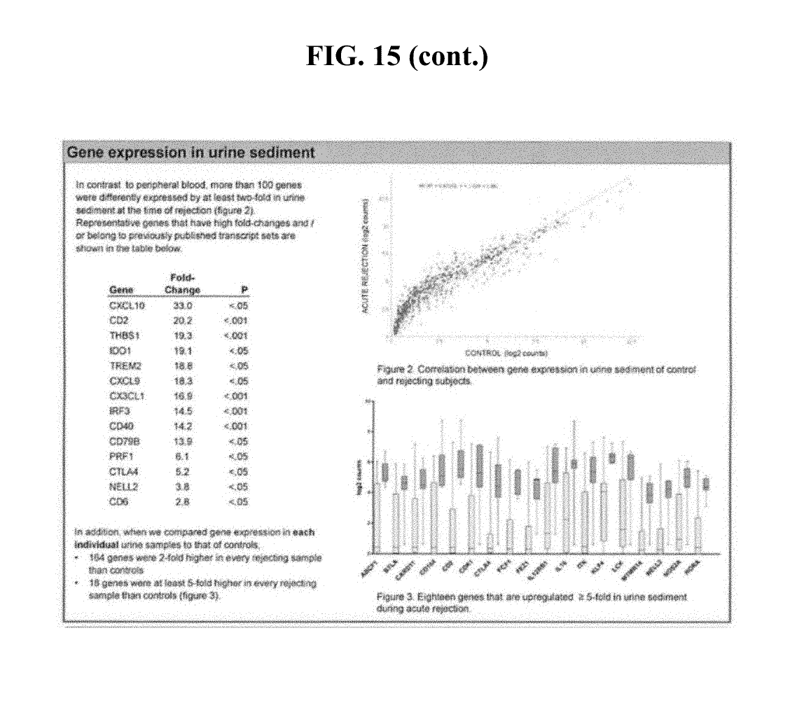

We compared gene expression levels in each individual acute rejection sample to that of the average expression of all the control samples to identify genes that changed at least 2-fold in every rejecting subject. In peripheral blood RNA, 63-239 genes were upregulated >2-fold in individuals experiencing rejection and 27-298 genes were downregulated (See FIG. 15). However, there were no common genes that increased >2-fold in all of the 7 rejection samples. In individual urine sediment RNA from patients experiencing rejection, expression of 250-720 genes were increased >2-fold, 86-590 were increased >5-fold and 30-370 were Increased >10 fold when compared to expression of the genes in urine from controls (see FIG. 15). Importantly, expression of 164 of these genes was increased >2-fold and expression of 18 was increased >5-fold in all of the rejection patient urine samples (see FIG. 15). The common genes included those in apoptosis, T cell activation and inflammatory pathways. No genes were observed to be down-regulated In urine at the time of acute rejection vs. controls.

The results of this Initial study indicate the power of the NanoString platform to measure gene expression changes in the urine that are associated with ongoing acute rejection in kidney transplants. These changes are not as substantially evident when measured in peripheral blood. This approach has the potential to develop a multi-gene signature for non-invasiveness diagnosis of rejection.

An additional trial was conduct called CTOT 19. A primary goal of the Molecular Core for CTOT-19 is to develop molecular signatures for diagnosis and provision of molecular mechanistic insights in kidney grafts for the patients enrolled in CTOT-19. Our efforts have focused on the development of noninvasive approaches, primarily interrogating urine of the transplant patients as a source of biomarker mRNAs.

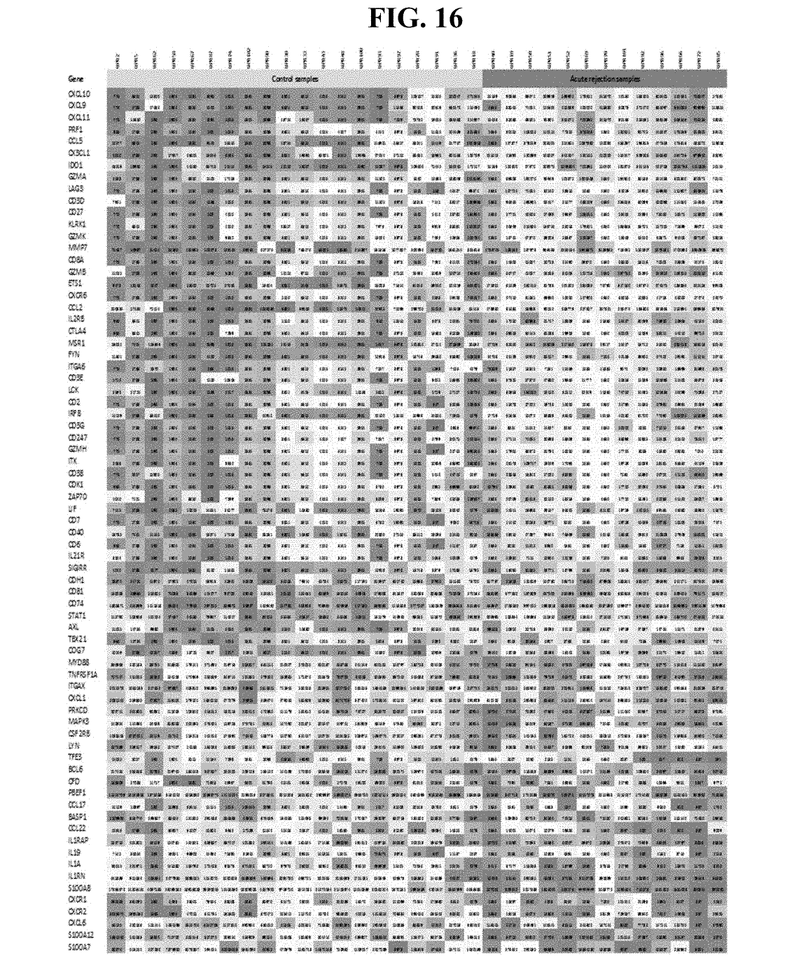

Our initial studies on urine sediment RNA from patients in CTOT-16 tested RNA isolated from urine sediments of 13 kidney transplant patients at the time they experienced acute graft rejection and RNA isolated from the urine sediments of 20 kidney transplant patients not experiencing acute rejection (i.e. control samples) for the expression of 795 inflammatory and immune function genes (See FIG. 16). In NanoString analysis of urine RNA samples from patients experiencing graft rejection, a total of 161 genes are upregulated and 51 genes are down regulated vs. control levels at the time of acute rejection by 2 fold or greater. We found highly significant differences in gene expression between urine RNA from rejecting and non-rejecting control subjects (FIG. 16, volcano plot-upregulated genes during rejection on the right and down-regulated on the left). The approach distinguishes rejection from control/non-rejecting samples with expression of some inflammatory genes having as much as 30-fold increases and down-regulation of tissue homeostasis genes vs. controls (FIG. 16, heat map). We used logistic regression and area under the curve (AUC) analysis of this data to derive an equation that provide a cutoff for the best set of genes distinguishing kidney graft rejection vs. no rejection with an AUC of 0.824; 95% confidence intervals of 0.7773-0.8707, p<0.0001. From these initial studies, values over vs. under the AUC clearly distinguish the absence vs. presence of kidney graft rejection.

As an extension of these studies we tested the ability to distinguish acute rejection from inflammation caused by BK/UTI using the urine RNA samples from rejecting patient with ongoing acute rejection and a new set of urine RNA samples from 10 patients with well-functioning kidney grafts but with BK virus infections. The results indicate the clear ability to distinguish between these two types of inflammatory insults with the AUC of the comparative analyses >0.960. This in itself is an advance in providing a rapid and cheap approach to distinguishing between these two types of inflammatory insults to kidney grafts by interrogating the urine.

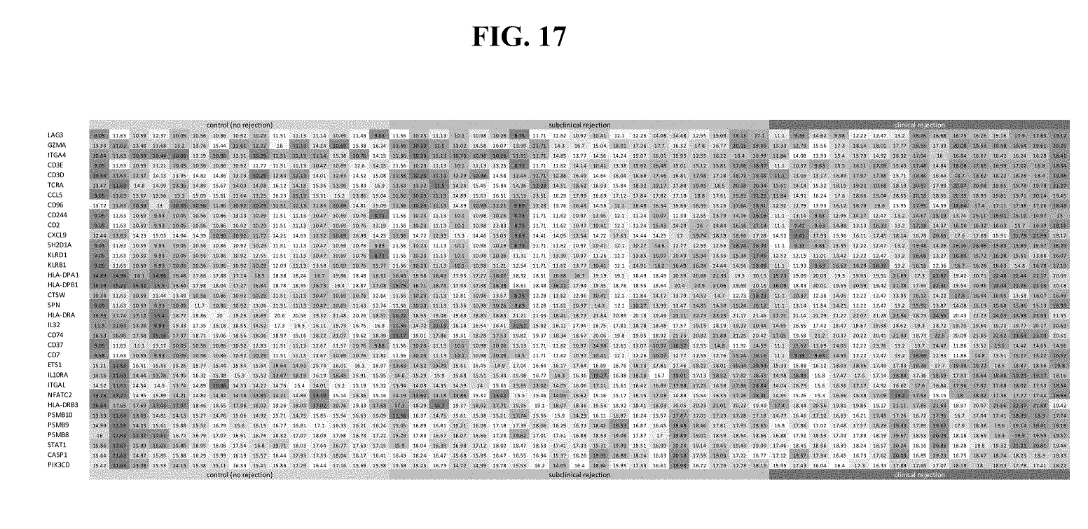

Finally, we obtained approximately 560 frozen urine sediments collected during the CTOT-08 study from Northwestern University. These sediments were collected serially from 100 patients that included samples obtained at the time of biopsy-proven acute (clinical) rejection, biopsy diagnosed subacute rejection, and no graft injury. RNA was purified from all of the samples and interrogated by the NanoString as above but this time in a blinded manner. The results (see FIG. 17) indicated that the gene expression profile (11 genes) indicating ongoing acute rejection in the initial study did not indicate ongoing acute rejection in the CTOT-08 study. However, the results of the CTOT-08 samples were reanalyzed and a distinct gene expression profile for ongoing acute rejection was evident, only different from that observed in the initial studies (Heat Map FIG. 17 below). It is important to note that the sample from the initial study were obtained from patients enrolled in the Belatacept trial in CTOT-15/-16, whereas the recipients in CTOT-08 were treated with campath and maintained on tacrolimus. This suggests that the gene expression profiles indicating ongoing acute rejection may be different depending on the immunosuppression strategy used for the transplant patient. Another important finding from this data is that the samples from patients at the time sub-clinical rejection was diagnosed fall into one of two distinct gene expression patterns; one similar to that observed when there is no rejection and the other closer to that observed during ongoing clinical rejection. The graft outcomes for each of these two groups will have to be assessed to determine if the two gene expression profiles are prognostic of development to more severe graft injury or not.

### REFERENCES

1. Keslar et al., Am. J. Transplant, 13:1891-1897, 2013.

2. Hricik et al., Am. J. Transplant, 13:2634-2644, 2013.

3. Suthanthiran et al., N. Engl. J. Med., 369:20-31, 2013.

4. Hricik et al., J. Am. Soc. Nephrol., 26:3114-3122, 2015.

5. WO2015200873

6. WO2013173493.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A method of detecting rejection, or an elevated risk of rejection, of an organ or tissue graft in a subject comprising:
detecting an upregulated level of at least one first RNA marker in a sample from a subject and/or a downregulated level of at least one second RNA marker in said sample from said subject, and thereby detecting acute rejection and/or elevated risk of rejection of said organ or tissue graft,
wherein said sample comprises urine from said subject,
wherein said subject has a tissue or organ graft,
wherein said at least one first RNA marker that is detected as being upregulated is selected from the group consisting of: CXCL10, CXCL9, CXCL11, PRF1, CCL5, CX3CL1, IDO1, GZMA, LAG3, CD3D, CD27, KLRK1, GZMK, MMPI, CD8A, GZMB, ETS1, CXCR6, CCL2, IL2RB, CTLA4, MSR1, FYN, ITGA6, CD3E, LCK, CD2, IRF8, CD3G, CD247, GZMH, ITK, CD38, CDK1, ZAP70, LIF, CD7, CD40, CD6, IL21R, SIGIRR, CDH1, CD81, CD74, STAT1, AXL, TBX21, COG7, TCRA, C1QB, NKG7, THBS1, C1QA, EPCAM, DPP4, HLA-DPA1, TIGIT, HLA-DPB1, CXCL13, C1R, CD5, GPR171, EFEMP1, HLA-DMA, LY9, CCL8, CD96, HAVCR2, NFATC2, LAMC2, IFNG, STAT4, APOE, C4B, CTSL, SOCS1, PVR, CCR5, CD86, TNFSF12, SIGLEC1, ST6GAL1, C3, KLRC2, CTSW, CARD11, GNLY, PSMB10, ICOS, TAP1, TNFRSF11B, ITGA1, IRF4, CD4, CD84, CSF1, SH2D1A, CD8B, CD47, ABCB1, SLAMF1, HLA-DMB, IFI35, A2M, HLA-DRA, C1QBP, SPP1, VCAM1, NELL2, FLT3LG, ITGA4, TNFRSF10B, RUNX3, SERPING1, KLRC1, NLRC5, HPRT1, IKBKE, TNF, ADA PDCD1, C 1 S, CCR2, TAB1, IL12RB1, IL2RA, PSEN2, LRP1, NOL7, SPN, TCF7, MFGE8, MST1R, IL10, LRRN3, PSMB9, PPIA, AGK, EDC3, CD80, PSMB8, CD276, BIRC5, TNFRSF9, TP53, ZC3H14, HLA-DRB3, ICOSLG, CD200, PIN1, EGR2, ITGAE, TRAF2, ABL1, ALCAM, IRF5, SF3A3, HRAS, NOD1, PDCD1LG2, IL16, LILRB1, and IRF3; and
wherein said at least one second RNA marker that is detected as being downregulated is selected from the group consisting of: ANXA1, BASP1, BCL6, BLK, CAMP, CCL17, CCL22, CCR3, CEACAM1, CEACAM6, CFD, CR2, CSF2RB, CSF3R, CXCL1, CXCL6, CXCR1, CXCR2, FUT7, IFNL1, IL18RAP, IL19, ILIA, IL1RAP, IL1RN, IL8, ITGAX, LCN2, LILRB3, LYN, MAPK3, MEFV, MYD88, OSM, PBEF1, POLR2A, PRKCD, PTGS2, S100Al2, S100A7, S100A8, SH2B2, SH2D1B, SPINK5, TFE3, TLR1, TNFRSF 10C, TNFRSF11A, TNFRSF1A, TNFSF14, ADAMS, ARG2, BCL10, CASP3, CD46, CFI, DMBT1, DOCKS, DUSP4, IFI27, IKBKB, IL23A, ISG15, ITCH, LGALS3, MAPK8, MUC1, NOS2A, SERPINA3, SERPINB2, SYT17, and TREM1.

2. The method of claim 1, wherein said subject was taking a first immunosuppressant at the time said sample was obtained.

3. The method of claim 2, further comprising the step of performing at least one of the following:
i) treating said subject with a second immunosuppressant that is different from said first immunosuppressant;
ii) generating and/or transmitting a report that indicates said at least one first RNA maker is upregulated and/or said at least one second RNA marker is downregulated in said sample, and/or that said subject is in need of an immunosuppressant different from said first immunosuppressant, and/or is in need of a having said tissue and/or organ graft removed or replaced;
iii) generating and/or transmitting a report that indicates said at least one first RNA maker is upregulated and/or said at least one second RNA marker is downregulated in said sample, and/or that said subject has acute rejection and/or elevated risk of rejection of said organ or tissue graft; and
iv) characterizing said subject as having acute rejection and/or an elevated risk of rejection of said organ or tissue graft, based on finding that said at least one first RNA maker is upregulated and/or said at least one second RNA marker is downregulated in said sample.

4. The method of claim 1, wherein said organ or tissue graft comprises a kidney or kidney tissue graft.

5. The method of claim 1, wherein said subject at the time of collection of said sample was being treated with, and/or wherein said sample comprises, an immunosuppressant protein that comprises an extracellular domain of CTLA-4.

6. The method of claim 5, wherein said immunosuppressant protein further comprises an Fc region.

7. The method of claim 5, wherein said immunosuppressant protein comprises Belatacept or Abatacept.

8. The method of claim 1, wherein said detecting at least one of said first and/or second RNA marker comprises detecting at least two of said first and/or second RNA markers.

9. A composition comprising:
a) a sample from a subject having a tissue or organ graft, wherein said sample comprises urine from said subject; and
b) reagents configured for detecting the level of at least one RNA marker selected from the group consisting of: CXCL10, CXCL9, CXCL11, PRF1, CCL5, CX3CL1, IDO1, GZMA, LAG3, CD3D, CD27, KLRK1, GZMK, MMPI, CD8A, GZMB, ETS1, CXCR6, CCL2, IL2RB, CTLA4, MSR1, FYN, ITGA6, CD3E, LCK, CD2, IRF8, CD3G, CD247, GZMH, ITK, CD38, CDK1, ZAP70, LIF, CD7, CD40, CD6, IL21R, SIGIRR, CDH1, CD81, CD74, STAT1, AXL, TBX21, COG7, TCRA, C1QB, NKG7, THBS1, C1QA, EPCAM, DPP4, HLA-DPA1, TIGIT, HLA-DPB1, CXCL13, C1R, CD5, GPR171, EFEMPL HLA-DMA, LY9, CCL8, CD96, HAVCR2, NFATC2, LAMC2, IFNG, STAT4, APOE, C4B, CTSL, SOCS1, PVR, CCRS, CD86, TNFSF12, SIGLEC1, ST6GAL1, C3, KLRC2, CTSW, CARD11, GNLY, PSMB10, ICOS, TAP1, TNFRSF11B, ITGA1, IRF4, CD4, CD84, CSF1, SH2D1A, CD8B, CD47, ABCB1, SLAMF1, HLA-DMB, IFI35, A2M, HLA-DRA, C1QBP, SPP1, VCAM1, NELL2, FLT3LG, ITGA4, TNFRSF10B, RUNX3, SERPING1, KLRC1, NLRC5, HPRT1, IKBKE, TNF, ADA PDCD1, C1S, CCR2, TAB1, IL12RB1, IL2RA, PSEN2, LRP1, NOL7, SPN, TCF7, MFGE8, MST1R, IL10, LRRN3, PSMB9, PPIA, AGK, EDC3, CD80, PSMB8, CD276, BIRC5, TNFRSF9, TP53, ZC3H14, HLA-DRB3, ICOSLG, CD200, PIN1, EGR2, ITGAE, TRAF2, ABL1, ALCAM, IRF5, SF3A3, HRAS, NOD1, PDCD1LG2, IRF3, ANXA1, BASP1, BCL6, BLK, CAMP, CCL17, CCL22, CCR3, CEACAM1, CEACAM6, CFD, CR2, CSF2RB, CSF3R, CXCL1, CXCL6, CXCR1, CXCR2, FUT7, IFNL1, IL18RAP, IL19, IL1A, IL1RAP, IL1RN, IL8, ITGAX, LCN2, LILRB3, LYN, MAPK3, MEFV, MYD88, OSM, PBEF1, POLR2A, PRKCD, PTGS2, S100Al2, S100A7, S100A8, SH2B2, SH2D1B, SPINKS, TFE3, TLR1, TNFRSF10C, TNFRSF11A, TNFRSF1A, TNFSF14, ADAMS, ARG2, BCL10, CASP3, CD46, CFI, DMBT1, DOCKS, DUSP4, IFI27, IKBKB, IL23A, ISG15, ITCH, LGALS3, MAPK8, MUC1, NOS2A, SERPINA3, SERPINB2, SYT17, and TREM1.

10. The compositing of claim 9, wherein said urine sample is from a subject having a kidney tissue or organ graft.

11. The composition of claim 9, wherein said subject at the time of collection of said sample was being treated with, and/or wherein said sample comprises, an immunosuppressant protein that comprises an extracellular domain of CTLA-4.

12. The composition of claim 11, wherein said immunosuppressant protein further comprises an Fc region.

13. The composition of claim 12, wherein said immunosuppressant protein comprises Belatacept or Abatacept.

14. A method of detecting rejection, or an elevated risk of rejection, of an organ or tissue graft in a subject comprising:
detecting an upregulated level of at least one first RNA marker in a sample from a subject and/or a downregulated level of at least one second RNA marker in said sample from said subject, and thereby detecting acute rejection and/or elevated risk of rejection of said organ or tissue graft,
wherein said sample comprises urine from said subject,
wherein said subject has a tissue or organ graft,
wherein said at least one first RNA marker that is detected as being upregulated is selected from the group consisting of: TNFRSF11B, ETS1, CXCL10, CDK1, CXCL9, SIGIRR, CD274, CXCL11, ITGA6, and SF3A3, and
wherein said at least one second RNA marker that is detected as being downregulated, are selected from the group consisting of: TNFSF4, ATG16L1, MERTK, DHX16, CXCL14, IL19, CCL17, ATF1, CFD, IFIT1, CD163, ELK1, DPP4, and LAMP2.

15. The method of claim 14, wherein said subject was taking a first immunosuppressant at the time said sample was obtained.

16. The method of claim 15, further comprising the step of performing at least one of the following:
i) treating said subject with a second immunosuppressant that is different from said first immunosuppressant;
ii) generating and/or transmitting a report that indicates said at least one first RNA maker is upregulated and/or said at least one second RNA marker is downregulated in said sample, and/or that said subject is in need of an immunosuppressant different from said first immunosuppressant, and/or is in need of a having said tissue and/or organ graft removed or replaced;
iii) generating and/or transmitting a report that indicates said at least one first RNA maker is upregulated and/or said at least one second RNA marker is downregulated in said sample, and/or that said subject has acute rejection and/or elevated risk of rejection of said organ or tissue graft; and
iv) characterizing said subject as having acute rejection and/or an elevated risk of rejection of said organ or tissue graft, based on finding that said at least one first RNA maker is upregulated and/or said at least one second RNA marker is downregulated in said sample.

17. The method of claim 14, wherein said organ or tissue graft comprises a kidney or kidney tissue graft.

18. The method of claim 14, wherein said subject has been treated with an anti-CD52 monoclonal antibody or antigen binding fragment thereof.

19. The method of claim 18, wherein said anti-CD52 monoclonal antibody comprises Alemtuzumab/CAMPATH.

20. The method of claim 14, wherein said subject at the time of collection of said sample was being treated with, and/or wherein said sample comprises, an immunosuppressant comprising tacrolimus (aka FK506).

21. The method of claim 14, wherein said detecting at least one of said first and/or second RNA marker comprises detecting at least two of said first and/or second RNA markers.

22. The method of claim 14, wherein said detecting at least one of said first and/or second RNA marker comprises detecting at least four of said first and/or second RNA markers.

23. A composition comprising:
a) a sample from a subject having a tissue or organ graft, wherein said sample comprises urine from said subject; and
b) reagent configured for detecting the level of at least one RNA marker selected from the group consisting of: CCL17, CD163, CD274, CDK1, CFD, CXCL10, CXCL11, CXCL14, CXCL9, DHX16, DPP4, ELK1, ETS1, IFIT1, IL19, ITGA6, LAMP2, MERTK, SF3A3, SIGIRR, TNFRSF11B, and TNFSF4.

24. The compositing of claim 21, wherein said urine sample is from a subject having a kidney tissue or organ graft.

25. The composition of claim 21, wherein said subject has been treated with an anti-CD52 monoclonal antibody or antigen binding fragment thereof.

26. The composition of claim 25, wherein said anti-CD52 monoclonal antibody comprises Alemtuzumab/CAMPATH.

27. The composition of claim 23, wherein said subject at the time of collection of said sample was being treated with, and/or wherein said sample comprises, an immunosuppressant comprising tacrolimus (aka FK506).

28. A method of detecting rejection or BK virus infection, or an elevated risk of rejection, of an organ or tissue graft in a subject comprising:
detecting an upregulated level of at least one first RNA marker in a sample from a subject and/or a downregulated level of at least one second RNA marker in said sample from said subject, and thereby detecting acute rejection and/or elevated risk of rejection of said organ or tissue graft,
wherein said sample comprises urine from said subject,
wherein said subject has a tissue or organ graft,
wherein said at least one first RNA marker that is detected as being upregulated and is selected from the group consisting of: SIGLEC1, CD163, PSEN1, DPP4, CEACAM8, PYCARD, MTMR14, ICOSLG, NOL7, C2, HLA.DRB3, CD68, ST6GAL1, SLAMF1, MERTK, PRAME, ELK1, STAT4, CD99, CD86, LAMP2, ITGB1, IL21R, IL6ST, TNF, GPATCH3, GPI, ATF1, PIN1, and YTHDF2, and
wherein said at least one second RNA marker that is detected as being downregulated, is selected from the group consisting of: HMGB1, MAPK9, SPA17, TNFRSF11B, GTF3C1, BST2, PRPF38A, MAF, BATF, EGFR, CD274, CCL17, SERPINA3, GBP1, and PBEF1.

29. The method of claim 28, wherein BK virus infection is detected.

30. The method of claim 28, wherein rejection is detected.

31. The method of claim 28, wherein said organ or tissue graft comprises a kidney or kidney tissue graft.

32. The method of claim 31, wherein said subject has been treated with an anti-CD52 monoclonal antibody or antigen binding fragment thereof.

33. The method of claim 32, wherein said anti-CD52 monoclonal antibody comprises Alemtuzumab/CAMPATH.

34. The method of claim 28, wherein said subject at the time of collection of said sample was being treated with, and/or wherein said sample comprises, an immunosuppressant comprising tacrolimus (aka FK506).

35. A composition comprising:
a) a sample from a subject having a tissue or organ graft, wherein said sample comprises urine from said subject; and
b) reagent configured for detecting the level of at least one RNA marker selected from the group consisting of: ATF1, BATF, BST2, C2, CCL17, CD163, CD274, CD68, CD86, CD99, CEACAM8, DPP4, EGFR, ELK1, GBP1, GPATCH3, GPI, GTF3C1, HLA.DRB3, HMGB1, ICOSLG, IL21R, IL6ST, ITGB1, LAMP2, MAF, MAPK9, MERTK, MTMR14, NOL7, PBEF1, PIN1, PRAME, PRPF38A, PSEN1, PYCARD, SERPINA3, SIGLEC1, SLAMF1, SPA17, ST6GAL1, STAT4, TNF, TNFRSF11B, and YTHDF2.

36. The compositing of claim 35, wherein said urine sample is from a subject having a kidney tissue or organ graft.

37. The composition of claim 35, wherein said subject has been treated with an anti-CD52 monoclonal antibody or antigen binding fragment thereof.

38. The composition of claim 35, wherein said anti-CD52 monoclonal antibody comprises Alemtuzumab/CAMPATH.

39. The composition of claim 35, wherein said subject at the time of collection of said sample was being treated with, and/or wherein said sample comprises, an immunosuppressant comprising tacrolimus (aka FK506).
